(19) United States
(12) Patent Application Publication
(10) Pub. No.: US 2018/0169205 A1
(43) Pub. Date: Jun. 21, 2018

(54) HYBRID FLAGELLIN AS A T CELL INDEPENDENT VACCINE SCAFFOLD

(71) Applicant: The Regents of the University of California, Oakland, CA (US)

(72) Inventors: David D. Lo, Del Mar, CA (US); Kaila M. Bennett, Twin Peaks, CA (US)

(73) Assignee: The Regents of the University of California, Oakland, CA (US)

(21) Appl. No.: 15/737,058

(22) PCT Filed: Aug. 6, 2016
(86) PCT No.: PCT/US16/45929

§ 371 Date: Dec. 15, 2017

Related U.S. Application Data
(60) Provisional application No. 62/202,054, filed on Aug. 6, 2015.

(51) Int. Cl. A61K 39/02; A61K 39/12; A61K 39/39; C07K 2/00

(52) U.S. Cl. CPC A61K 39/02; A61K 39/12; A61K 2039/54; C07K 2/00; A61K 39/39

(57) ABSTRACT

A method of immunizing a subject. The method includes administering to the subject an immunogenic amount of a filament including one or more hybrid flagellin proteins, each hybrid protein including a heterologous polypeptide antigen and a portion of a FliC flagellin protein. As a result of the administration, a TI immune response to the polypeptide antigen can be produced in the subject.

### CROSS-REFERENCE TO RELATED APPLICATIONS

This application claims the benefit of Provisional Patent Application No. 62/202,054, filed on Aug. 6, 2015, which is incorporated by reference herein.

### STATEMENT REGARDING FEDERALLY SPONSORED RESEARCH OR DEVELOPMENT

This invention was made with Government support under Grant Nos. AI063426 and AI082185 awarded by the National Institutes of Health, and Grant No. 2011119474 awarded by the National Science foundation. The Government has certain rights in this invention.

### BACKGROUND

### Field of the Invention

The invention relates to vaccines.

### Related Art

Adaptive immune antibody responses rely primarily on the interaction between T helper cell populations promoting B cell activation, isotype switching, and development of plasma cells producing immunoglobulin, with subsequent pathogen opsonization/viral neutralization [1-3]. However, in T cell-deficiency settings such as AIDS, this TD mode of antibody production is ineffective at conferring protection to the host. However, “innate-like” B cells, which include splenic marginal zone B cells (15% of total B cells) and peritoneal B1 B cells (B1a and B1b, 3% of total of B cells), can become activated after recognition of repetitive or polymeric antigenic epitopes causing B cell receptor (BCR) clustering, independently of T helper (Th) subset (TI: [4,5]). Efficient BCR clustering is crucial for the generation of a TI antibody response and in fact it has been determined that 10-20 BCRs must be crosslinked in order to mount a TI response [6,7]. On resting B cells, BCRs are spaced approximately 35 nm apart, requiring TI antigens to be at least 500 nm in length [7]. Despite the chemical diversity of TI antigens, most TI antigens repetitive epitopes are spaced 5-10 nm [6,8]. Once a TI B cell has become activated, its antibody response is largely characterized by the production of IgM and certain IgG isotypes (IgG3 and possibly IgG1: [9]). B1 B cells have also been found to undergo class switch recombination from IgM to IgA. Although the anatomic site of this isotype switch remains elusive, this suggests that a significant proportion of IgA may be generated in a TI manner [10].

While many bacterial capsular polymeric components have been studied for their ability to stimulate a TI antibody response, it remained to be tested whether bacterial flagellin (FliC), a major antigen present on many commensal and pathogenic microbes, can be exploited as a scaffold for TI vaccines [11,12]. Indeed, one study suggested that polymeric flagellin, like its monomeric form, relies on T cell help to stimulate a humoral immune response. However, this study did not include IgM and IgG3 (two major antibody isotypes indicative of TI response) in their analysis [13]. FliC from Salmonella serovar enterica typhimurium (S. Typhimurium) is the major protein making up the flagellum (11-fold symmetry) and can grow to 15 μm in length [14]. In vitro studies revealed that FliC is able to form filaments hundreds of nanometers in length [15]. This is a critical feature for the success of TI vaccines, as it has been suggested that a TI antigen needs to be at least 500 nm in length to produce a significant TI response.

FliC is a four-domain (D0, D1, D2, D3) protein that structurally resembles the Greek letter Γ (FIG. 1), and has been well characterized for its adjuvant activity due to its Toll-like receptor 5 (TLR5) and Nod-like receptor CARD domain-containing protein 4 (NLRC4) binding sites located in its D1 domain, a domain also responsible for polymerization [14, 16-18]. In polymerized FliC filaments, the D3 domain of FliC is completely solvent exposed, while other domains remain mostly buried within the inner core. Moreover, based on the crystallographic structure of the Filament the D3 domains are spaced ˜5 nm apart. Muskotal et al. [19] found that as a monomer the D3 domain is the most stable portion of FliC, where D0 and D1 completely lose their α-helical secondary structure, corroborating previous structural studies. Moreover, by eliminating D3, this study found that D3 was dispensable for the overall stability of the flagellum filament, marking D3 as a possible insertion site for novel antigenic determinants.

### SUMMARY

To extend the potency of vaccines against infectious diseases, vaccines should be able to exploit multiple arms of the immune system. One component of the immune system that is under-used in vaccine design is the subset of B cells known to be capable of responding to repetitive antigenic epitopes and differentiate into plasma cells even in the absence of T cell help (T-independent, TI).

Thus, in one aspect, a method of inducing a T cell-independent (TI) immune response in a subject is provided. The method includes administering to the subject an immunogenic amount of a filament comprising one or more hybrid flagellin proteins, each hybrid protein comprising a heterologous polypeptide antigen and a portion of a FliC flagellin protein, whereby a TI immune response is generated against the antigen.

In the method: a) the antigen can replace a whole or a part of FliC flagellin protein variable domain D3; b) the antigen can comprise an amino acid sequence from a pathogenic virus; c) the antigen can comprise a protective epitope; d) the filament can comprise two or more hybrid flagellin proteins, each hybrid protein comprising a different heterologous polypeptide antigen, which can be a different serotype of a pathogenic virus; e) the antigen can be a viral envelope protein; f) the FliC flagellin protein can be from S. Typhimurium; g) the TI immune response can include the production of IgM and/or IgG3 antibody; h) a T cell-dependent (TD) response can also be generated against the antigen; i) the subject can be a human or other mammal; j) subject can be immunocompromised, elderly, or a neonate, or any combination thereof; k) an adjuvant can administered with the hybrid flagellin protein; or l) any combination of a)-k).

In another aspect, a vaccine composition is provided. The composition includes an immunogenic amount of a filament comprising one or more hybrid flagellin proteins, each hybrid protein comprising a heterologous polypeptide antigen and a portion of a FliC flagellin protein. In the composition: a) the antigen can replace a whole or a part of FliC flagellin protein variable domain D3; b) the antigen can comprise an amino acid sequence from a pathogenic virus; c) the antigen can comprise a protective epitope; d) the filament can comprise two or more hybrid flagellin proteins, each hybrid protein comprising a different heterologous polypeptide antigen, which can be a different serotype of a pathogenic virus; e) the antigen can be a viral envelope protein; f) the FliC flagellin protein can be from S. Typhimurium; g) the composition can include an adjuvant; or h) any combination of a)-g).

In a further aspect, an isolated nucleic acid encoding the hybrid protein is provided. Also provided is an expression vector (or more generally any nucleic acid vector) comprising the isolated nucleic acid, and a host cell containing the expression vector.

### BRIEF DESCRIPTION OF THE DRAWINGS

For a more complete understanding of the present invention, reference is now made to the following descriptions taken in conjunction with the accompanying drawings, in which:

FIG. 1A shows schematic representations of a hybrid flagellin protein.

FIG. 1B is a panel showing a gel and Western blot of a purified hybrid flagellin protein.

FIG. 1C shows circular dichroism spectra of various proteins including hybrid flagellin protein.

FIG. 2A is a panel of molecular structures and transmission electron (TE) micrographs for native and hybrid flagellin proteins.

FIG. 2B shows data characterizing hybrid flagellin filaments.

FIG. 2C shows additional data characterizing hybrid flagellin filaments.

FIG. 3 shows features of a non-restricted mouse model.

FIG. 4 shows that hybrid flagellin filaments are able to crosslink the B cell receptor (BCR) in vitro.

FIG. 5 shows features of a TI mouse model.

FIG. 6 shows data characterizing hybrid flagellin filaments.

FIG. 7 shows TE micrographs for characterizing hybrid flagellin filaments.

FIG. 8 shows Western blots of antibody proteins.

FIG. 9 shows ELISA curves of antibodies from immunized mice.

FIG. 10 shows features of an experiment involving immunized B10.D2 mice.

### DETAILED DESCRIPTION

In the method of inducing a T cell-independent (TI) immune response in a subject, the hybrid protein comprises a portion of FliC flagellin protein and a heterologous polypeptide antigen.

The heterologous antigen includes non-Flic flagellin amino acid sequences. The heterologous antigen can include a whole or a portion of a polypeptide from an infectious agent or pathogen. In some embodiments, the antigen can include a combination of different polypeptides, or portions thereof, from an infectious agent or pathogen. In some embodiments, the antigen can include a combination of different polypeptides, or portions thereof, from different infectious agents or pathogens, or different serotypes of a particular infectious agent or pathogen. The antigen can be an antigen capable of inducing immunity and/or providing treatment for the subject. Thus, the antigen can comprise one or more protective epitopes from polypeptides such as virus envelope proteins, bacterial toxins, tumor antigens, parasite antigens, allergens, or other heterologous antigens.

Examples of polypeptides for inclusion in the heterologous antigen include, but are not limited to, Dengue 2 virus coat protein DENV2, virus coat protein from Zika virus or other flavivirus, virus coat protein from Ebola virus or other Filovirus, influenza hemagglutinin, HIV gp120, and a bacterial toxin such as diphtheria toxin.

The infections agent or pathogen can be, but is not limited to, a virus pathogen, a bacterial pathogen, a fungal pathogen, a protozoan parasite, or a helminth parasite.

The Flic flagellin protein can be a Salmonella protein, or a flagellin monomer from any flagellated bacterial pathogen such as Helicobacter.

In some embodiments, two or more hybrid flagellin proteins are provided, with each hybrid protein comprising the same or similar FliC flagellin protein sequence, but a different heterologous polypeptide antigen. The multiple hybrid proteins can then be combined into a single “mixed” filament.

The subject can be a human or another mammal, such as a horse, dog, cat, mouse, rat, and the like. The subject can be an immunologically normal individual, an immunocompromised individual (such as an AIDS patient or a person undergoing chemotherapy), an individual with relatively poor immune responses to vaccination (such as an elderly person or a neonate), or any combination thereof. The method can also be applied in cases where a conventional vaccine (e.g., flu vaccine) has a low response rate.

Exposure to filaments containing polymerized hybrid proteins can produce a T cell independent immune response, a T cell dependent immune response, or both, to the antigen. The immune response can provide protection and/or treatment for the subject against infectious agents, including pathogenic organisms.

Filaments comprising the hybrid flagellin protein can be administered as part of a vaccine composition. Thus, the filaments can be formulated with components such as additional adjuvants (e.g., CpG oligonucleotides), excipients or other components to aid in dispersal or administration (e.g., glycerol), or preservatives to help stabilize the vaccine in storage (e.g., chitosan). In a combination vaccine, two or more filaments comprising different hybrid flagellin proteins can be combined in a single vaccine and administered to the subject.

A variety of adjuvants known to one of ordinary skill in the art may be administered in conjunction with the filaments in an immunogen or vaccine composition. Such adjuvants include, but are not limited to polymers, co-polymers such as polyoxyethylene-polyoxypropylene copolymers, including block co-polymers, polymer p 1005, Freund's complete adjuvant (for animals), Freund's incomplete adjuvant; sorbitan monooleate, squalene, CRL-8300 adjuvant, alum, QS 21, muramyl dipeptide, CpG oligonucleotide motifs and combinations of CpG oligonucleotide motifs, trehalose, bacterial extracts, including mycobacterial extracts, detoxified endotoxins, membrane lipids, or combinations thereof.

The immunogenic or vaccine composition can be administered through different routes, such as oral or parenteral, including, but not limited to, buccal and sublingual, rectal, aerosol, nasal, intramuscular, subcutaneous, intradermal, and topical. The composition may be administered in different forms, including, but not limited to, solutions, emulsions and suspensions, microspheres, particles, microparticles, nanoparticles and liposomes. The amount, dosage and scheduling of immunizations will depend on the infection being targeted, the particular hybrid flagellin protein(s) being included in the immunization, the subject being treated, the manner of administration, and the like, which can be determined by those skilled in the art.

The immunogenic or vaccine composition can contain a pharmaceutically acceptable carrier or vehicle for administration to humans or animals. A pharmaceutically acceptable carrier or pharmaceutically acceptable vehicle is any composition or compound including, but not limited to, water or saline, a gel, salve, solvent, diluent, fluid ointment base, liposome, micelle, giant micelle, and the like. In general, the carrier or vehicle will be suitable for use in contact with living animal or human tissue, and does not interact with the other components of the composition in a deleterious manner.

Nucleic acids encoding the hybrid flagellin proteins can be prepared and expressed using standard molecular biology methods. Nucleic acid sequences encoding the FliC flagellin protein and a polypeptide antigen can be prepared, for example, by polymerase chain reaction, then expressed in a eukaryotic expression vector in a eukaryotic host cell. Purified hybrid flagellin protein monomers can be polymerized to form filaments for immunization or vaccine production.

In accordance with some embodiments, to target vaccine responses from T-independent B cells, a bacterial Flagellin was engineered (FliC) by replacing its exposed D3 domain with a viral envelope protein from Dengue virus (DENV2). The resulting hybrid FliC protein (hFliC) is able to form stable filaments decorated with conformationally intact DENV2 envelope domains. These filaments are not only capable of inducing a T cell-dependent (TD) humoral antibody response, but also significant IgM and IgG3 antibody response in a helper T cell repertoire-restricted transgenic mouse model. The NCBI Database Accession Number for S. Typhimurium FliC flagellin is NC_003197.1, and the GenBank Accession Number for DENV2 is JN796245.1; both sequences are incorporated by reference herein.

The following Examples provide information about these studies.

### Example 1

### Methods

Design of hFliC Protein

Flagellin was PCR amplified from an earlier vaccine design, where the DENV2 portion was linked to FliC at the N′ terminal domain through a GS linker. This FliC was first PCR amplified from genomic DNA isolated from a culture of S. Typhimurium. Flagellin was PCR amplified in two fragments. The first piece was amplified using a forward primer with flanking 5′ BamH1 site and reverse primer with 3′ flanking Kpn1, EcoR1 site, that had amplified only the first 184 residues. The second piece was amplified using a forward primer with 3′ flanking Kpn1, EcoRI site and reverse primer with 5′ Xho1 site, which amplified residues 285-494 plus a 6×-His tag. These two pieces were then PCR overlapped. DENV2 E sequence from a mouse adapted strain [39] was PCR amplified from the initial clone, and flanking N′ and C′ terminal GGGSGGGS linkers were then ligated into FliC lacking D3 via the Kpn1 and Ecor1 sites. Gene constructs were checked for appropriate sequence throughout the cloning process. This final construct was then ligated into the pENTR 3C (life technologies) expression vector for recombination and subsequent expression.

Production and Purification of Recombinant Proteins (hFliC, FliC and DENV2 E)

Recombinant protein was produced using Baculovirus expression system. First, cloned cDNA construct was transfected into SF21 insect cells (Life Technologies) in Grace's supplemented or unsupplemented media (Thermo Scientific) based on manufacturer's protocol. Resulting protein viral stocks were then transduced into SF9 insect cells (Life Technologies), cultured in HyClone SFX-Insect media (Thermo Scientific), for larger scale production. The insect cells secreting the protein were cultured at 27° C., and the supernatant was harvested after an optimized incubation period. The native FliC contains a signaling domain that allows the protein to be secreted, and this signaling domain was retained in the hFliC protein. As DENV2 E is a transmembrane protein, a leader peptide was added to it so that it would be secreted into the culture supernatant. The insect culture media was filtered before purification. Western blot (Life Technologies) was used to verify protein expression and purity before precipitating protein from the media with 80% saturated ammonium sulfate (Fisher). After a 2 hr precipitation at 4° C., protein was centrifuged at 12,000 rpm for 30 mins to pellet the protein. The protein pellet was resuspended with 1×PBS+0.05% Tween-20 and dialyzed over night in a porous membrane with a molecular weight cut off of 12-14,000 (SpectrumLabs). Protein was then bound to HisPur cobalt resin (Thermo Scientific) overnight. Bound protein was eluted from HisPur resin using increased concentrations of imidazole. Eluted protein was subjected to a final dialysis to rid the protein sample of any traces of Tween-20 and imidazole. The final protein was concentrated using polyethylene oxide (PEO: Sigma Aldrich). Concentrated protein was checked for purity using both Coomassie (Thermo Scientific) and Western blot (1° antibody rabbit-α-Ht (Santa Cruz Scientific) and 2° Donkey α-rabbit @680 nm (Odyssey)), and concentration was quantified either using BCA or Bradford.

Molecular Modeling of FliC and hFliC Filaments

The structure of FliC (PDB code 3A5X) and DENV2 (PDB code 1OAN) were obtained from the Protein Data Bank. The hFliC filament was built using the molecular structures of the FliC filament and the DENV2 protein. The D3 domain of a FliC monomer was deleted (residues 185-285), and the coordinates of DENV2 were manually translated and rotated until the N- and C-termini of DENV2 were in close proximity to the C- and N-termini of the end of the D2 domain of FliC. The linkers between FliC and DENV2 (sequence GGGSGGGS) were added using Modeller [40].

Polymerization of hFliC or FliC

Based on previous studies, purified FliC or hFliC proteins samples were diluted in either 3.0 M ammonium sulfate or sodium citrate [15,41]. A final buffer concentration of 1.5 M was obtained for each buffer+protein sample. This molar condition has been suggested to be ideal for FliC polymerization. Each buffer+protein sample was allowed to polymerize overnight at room temperature. Pelleted with high-speed centrifugation, and washed in with 150 mM PBS. These samples were then used for either TEM or vaccination.

### Circular Dichroism

Circular dichroism was used to determine secondary structure of the hFliC protein. Spectra were collected for each protein (FliC, DENV2, hFliC) using Jasco J-815 CD Spectrophotometer. Measurements were performed at 25° C. in a 1 cm path length cuvette (Varian), over a wavelength range of 190-260 nm. Protein for circular dichroism measurements was diluted in water to eliminate the effects of NaCl absorption, which absorbs strongly at wavelengths below 190 nm.

### Negative Stain Transmission Electron Microscopy

For transmission electron microscopy studies, formvar carbon coated grids (Ted Pella) were glow discharged for at least 20 seconds, 3 μl of polymeric sample was then loaded onto formvar carbon coated grid, and excess liquid was removed. Grids were then washed 3 times in PBS. For viewing in TEM, grids were negatively stained with either 4% uranyl acetate or 2% PTA (phosphotungsten acid) and placed into desiccator until imaged. Micrographs were obtained using TEM-1011 transmission electron microscope or PHILIPS TECNAI 12 transmission electron microscope.

### ELISA of Hybrid Flagellin

ELISAs were performed to see if the hFliC was capable of binding DENV2 specific antibodies. Briefly, ELISA plates were coated with either purified hFliC or purified FliC (negative control) at two different dilutions (1:2 and 1:4) and incubated overnight at 4° C. Plates were then washed, blocked and DENV2 specific monoclonal IgA antibody (at two dilutions) was added and again incubated overnight. Detection was performed by adding anti-IgA antibody (Southern Biotech) conjugated to Alkaline Phosphatase, and 4-MUP (Life Technologies) was added for detection by fluorescence.

### Design of DENV2 Recombinant IgA

We discovered that the 4G2 hybridoma produced 4 different Ig transcripts and multiple peptides, but only one pair specific for DENV2 E binding. Mass spectrometry N-terminal protein sequencing results of purified D1-4G2-4-15 hybridoma (4G2; ATCC# HB-112) antibody was used to determine the leader peptide nucleotide sequence of the correct variable regions. These 4G2 Dengue-specific kappa light and IgG2a heavy chain sequences were cloned and deposited in Genbank; NCBI accession code for kappa light chain is KJ438784 and IgG2a heavy chain is KJ438785. The full-length kappa sequence was amplified from 4G2 cDNA and cloned into pCDNA3.1(+) (Life Technologies) using NheI and EcoRI sites. The heavy chain variable region amplified from 4G2 hybridoma cDNA was overlapped with the IgA heavy constant region amplified from BALB/c spleen cell cDNA [22]. GS linker and 6×His-tag PCR fragment was added with the using another round of overlap PCR. The complete Dengue specific IgA heavy chain was cloned into pCAG-eGFP-puro by replacing the eGFP with Age1 and Not1 before ligation and transformation [42]. The Dengue specific IgA heavy chain clone was confirmed by sequencing.

### Production and Purification of DENV2 Recombinant IgA

Dengue specific kappa light and IgA heavy chain DNA constructs was stably transfected into the Chinese hamster ovary cell line (CHO; CHO-K1; ATCC# CCL-61) and clonally selected via limit dilution under selection media (F-12K media (Corning) supplemented with 10% fetal bovine serum (Biowest), 1000 μg/ml G418 (Corning), and 10 μg/ml puromycin (Sigma)). Final highest IgA-producing clone was adapted to serum free media (Corning) and used for production. The final clone was expanded in 40-T150-flasks and overgrown for one week prior to supernatant collection in serum free media. One liter of the supernatant media total protein was used to purify the rIgA (purification step as mentioned above). The production yield and purity was determined by Western blot, Coomassie, DENV2 protein binding ELISA, and total IgA ELISA quantification.

### Immunogold Labeling of Hybrid Flagellin Filaments

Immunogold labeling was performed to further confirm that the DENV2 subunit was presented on the surface of the filaments. Here, filaments were loaded onto carbon coated formvar grids. The grids were then incubated in 1×TBST (tris-buffered saline with Tween) with 0.05% BSA (Bovine serum albumin) for 30 minutes. Grids were washed and incubated with 4G2 (Dengue specific IgG hybridoma) for two hrs; again the grids were washed and incubated with Goat anti-mouse IgG conjugated gold particles (10 nm: Ted Pella). Finally, the grids were stained with 2% uranyl acetate and viewed.

### B Cell Crosslinking

Crosslinking studies were performed using a 4G2 B cell hybridoma (ATCC# HB-112). 4G2 cell line was cultured/maintained, based on ATCC recommendation, using Dulbecco's modified Eagle's medium (4 mM L-glutamine, 4.5 g/L of glucose, 1.5 g/L sodium bicarbonate, and 10% fetal bovine serum: Cellgro)). Cells were maintained at a concentration 1×10⁶ cells/ml, and were passed every three days. To assess if hFliC filaments were able to crosslink the BCRs of the 4G2 cell line, cells were first stimulated with a 20 μg dose of hFliC filaments (prepared as mentioned above) for 30 mins under ambient conditions. After the 30 mins incubation, cells were washed 3× in 1×PBS. Cells stimulated with hFliC filament antigen as unstimulated control were then spun onto glass slides (Superfrost plus, Fisher Scientific) using a Cytospin (Shandon). Cells were then fixed with 1% Paraformaldehyde/PBS and then permeabilized first with cytoskeleton buffer, followed by 0.5% Tween-20/PBS and washed with 3× 0.1% Tween-20/PBS. Cells were then blocked with 0.1% Tween-20 Casein solution (Thermo Scientific) for at least 30 mins. Cells were then stained with anti-Histag (Santa Cruz Biotechnology) polyclonal IgG antibody (@488 nm) for at least 1 hr, and again were washed. For cells stained with anti-histag antibody, these cells were again washed and secondary antibody was added (Donkey anti-rabbit @ 647: Life technologies) Finally, cells were post fixed with 4% PFA/PBS solutions and mounted with Prolong Gold antifade reagent (Life Technologies), which contained DAPI to counterstain the nuclei. Images were obtained using a spinning disk BD CARVII Confocal Imager (BD Biosystems) attached to a Zeiss Axio Observer inverted microscope. Hardware, including microscope, confocal and digital camera (Qimaging Rolera EMC²) was controlled using Metamorph imaging software. Images were further optimized using Velocity deconvolution software.

### Immunization Protocol

C57BL/6J, B10.D2, 6.5-TCR (Tg(Tcra/Tcrb)1Vbo) mice were maintained under specific pathogen free colony conditions at the UC Riverside vivarium. All procedures were performed in accordance with UCR Institutional Animal Care and Use Committee (IACUC) and NIH guidelines. Immunizations were performed over a five-week period, where the first dose (priming dose) was administered either through intranasal (IN) or intraperitoneal (IP) injections. For intranasal injections, a volume of 20 μl containing 20 μg protein was administered (10 μl containing 10 μg of protein into each nostril). For intraperitoneal injections, a volume of 200 μl, again with a total protein concentration of 20 μg, was injected into the peritoneum of mice using a 25 gauge needle. It should be noted all protein samples were diluted in 1×PBS to its final protein concentration. All mice were first anesthetized prior to injection. Three subsequent booster doses were given to all groups of mice via IN injections. Serum titers were assayed from peripheral blood collected by at the time points indicated. Mice were humanely sacrificed under anesthesia and cervical dislocation at the conclusion of the experiment.

### ELISA for Quantification of Antibody Titers

Black flat-bottom plates (Costar) were coated with 10 μg/ml recombinant DENV2 (prepared as described earlier), in coating buffer (25 mM Na₂CO₃, 75 mM NaHCO₃, pH 9.5). Plates were then washed 3× using 1×TBST buffer (50 mM Tris pH 7.5, 0.28 M NaCl, 6 mM KCl, 0.1% Tween 20), using a Biotek ELx405 Automated Plate Washer. Plates were blocked for 2 hrs at room temperature in 3% normal goat serum (Vector Labs). Samples were first diluted in blocking solution; serum was diluted 1:2000 and feces were diluted 1:10. Diluted samples were then two-fold serially diluted, and added to coated/blocked plated in triplicate. After washing, detection was performed with either Rat anti-mouse IgA-AP (Southern Biotech, diluted 1:1000), Goat anti-mouse IgM-AP (Southern Biotech, diluted 1:1000), Goat anti-mouse IgG-AP (Southern Biotech, diluted 1:2000), Goat anti-mouse IgG1-AP (Southern Biotech, diluted 1:1000), Goat anti-mouse IgG2A-AP (Southern Biotech, diluted 1:1000), Goat anti-mouse IgG2B-AP (Southern Biotech, diluted 1:1000), Goat anti-mouse IgG3-AP (Southern Biotech, diluted 1:1000), in 1×TBST. For final development, 10 mM 4-MUP (Molecular Probes) in DMSO diluted 1:25 in substrate buffer (50 mM K₂CO₃ 2 mM MgCl₂, pH 9.8) was added. Fluorescence was detected 60 minutes later 360 nm excitation and 460 nm emission wavelengths on Molecular Devices SpectraMax M2e plate reader. Raw fluorescence values from the dilution series were then subtracted from background fluorescence values.

### Statistical Tests

In general, groups were tested using Student's t-test, as indicated for each experiment in the figure legends, with a p<0.05 considered to be a significant difference for reporting. In the case of ELISA assays, Mann-Whitney tests were also used with similar results. In figures, *-p<0.05; **-p<0.005; ***-p<0.0005. Prism (GraphPad) software version 5.04 was used for statistical test calculations.

### Example 2

The polymeric nature of FliC filaments intrinsically provides the key feature needed for stimulating a potent TI response, so its ability as a vaccine scaffold for TI antigens was tested. The approach was to test the FliC D3 site for insertion of a fully independent conformational domain, and polymerizing the engineered protein as a vaccine target. Native FliC was engineered by replacing its entire D3 domain with the full length sequence of the Envelope glycoprotein (E) from Dengue virus serotype 2 (E), bridged by flexible linker sequences. This allowed for the antigen portion of the hybrid FliC (hFliC) protein to be fully exposed in polymerized filaments, enabling engagement of the antigen with the B cell antigen receptors for BCR crosslinking and subsequent TI stimulation. These studies showed that the hFliC filaments were able to stimulate a potent antibody response; more importantly, the filament induced a significant response in a helper T cell-restricted mouse model. Thus, the study presented herein describes the use of FliC as a novel scaffold for the design of potential TD/TI vaccines.

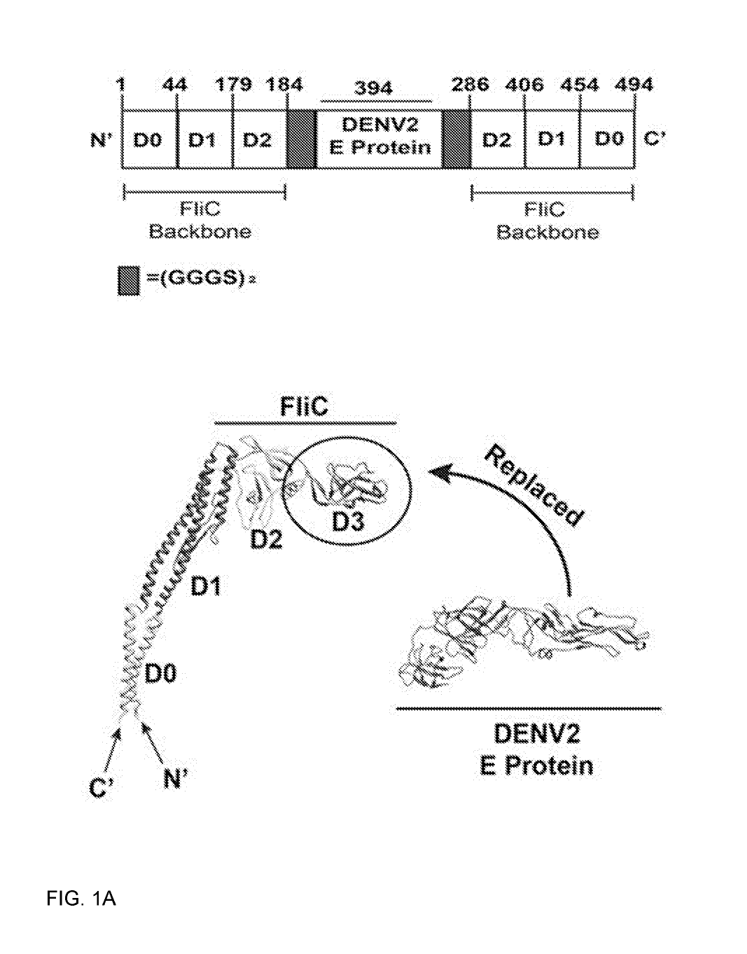

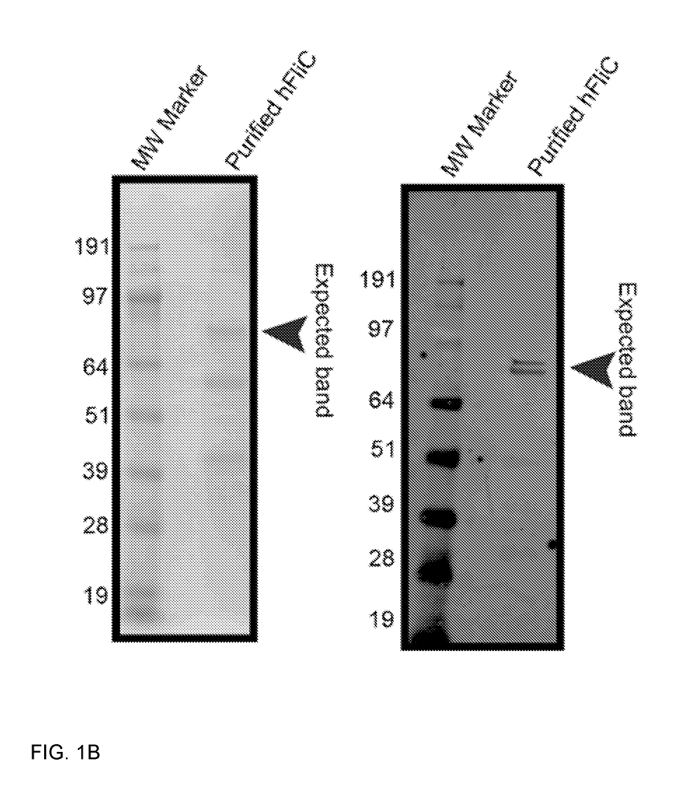

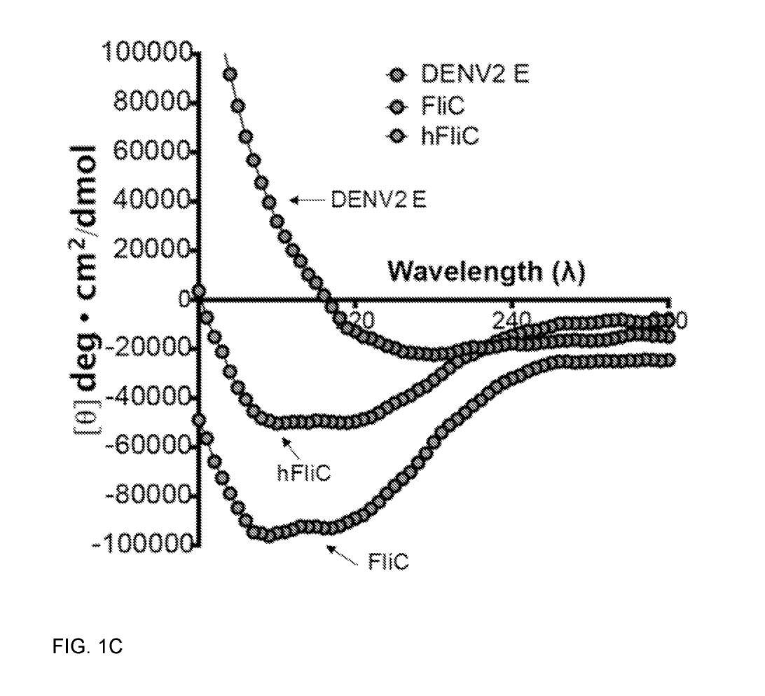

Referring to FIG. 1, this figure shows the design and analysis of hybrid flagellin protein. FIG. 1A is a schematic of the construction and design of hybrid flagellin (hFliC). The D3 domain from the native FliC protein was deleted (residues 185 to 285). DENV2 E plus GS linkers flanking the termini were inserted to replace the D3 domain of FliC. The final gene product was cloned into the pENTR plasmid for baculovirus expression of the hybrid protein. Referring to FIG. 1B, protein expressed by baculovirus expression system was checked for purity and anticipated molecular weight (˜85 kDa) using both Coomassie stain (left panel, which labels all proteins) and Western blot (right panel). In the Western blot, protein was probed with an antibody against the His-tag under denaturing conditions. Major bands from both blots indicate appropriate band size, however some degradation products were present in Coomassie gel; by densitometry the predicted hFliC band comprised approximately 15% of total protein. The double band in the Western blot suggests a proteolytic cleavage very near the N-terminus. Referring to FIG. 1C, circular dichroism (CD) was used to assess secondary structural feature of the hFliC protein. CD spectra of FliC (lower curve) exhibit a characteristic α-helical region, while DENV2 spectra (upper curve) exhibit a large β-sheet and coil regions. hFliC CD spectra (middle curve) was essentially a hybrid of the two proteins.

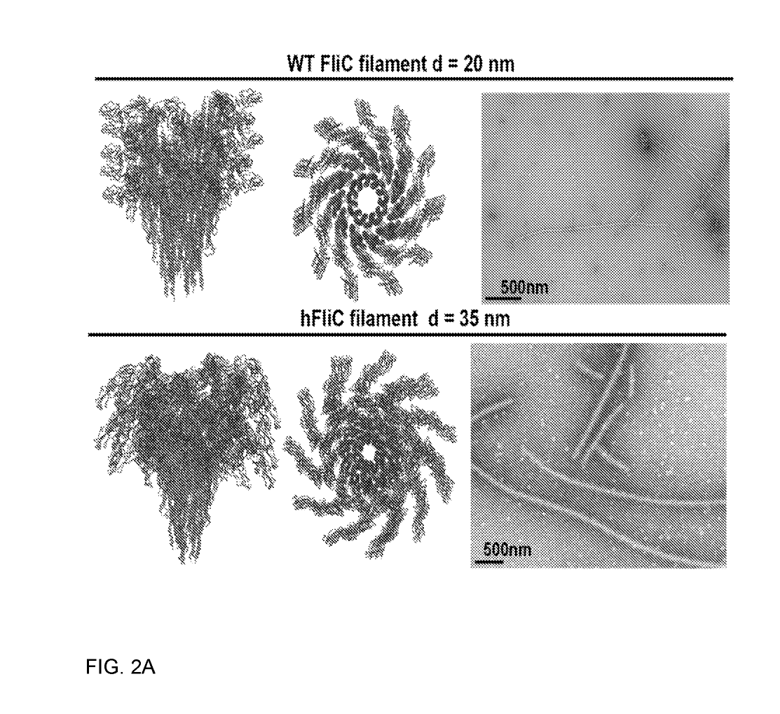

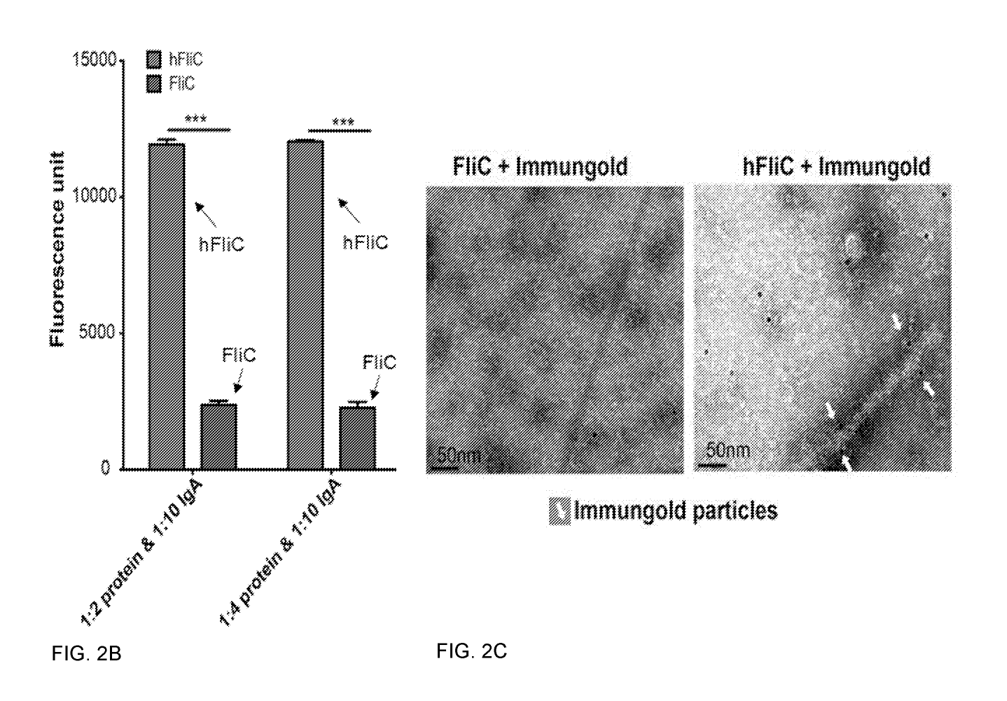

FIG. 2 is a characterization of hybrid flagellin filaments. In FIG. 2A, the top panel represents molecular structure of the parent FliC filament 13 (left side: XY view and middle: Z view). Negative stain TE micrograph of WT FliC filament generated by recombinantly produced FliC (right side); FliC filament diameter was calculated to be 20 nm. The bottom panel represents a hypothetical molecular model of hFliC, where DENV2+GS linkers were modeled into native FliC filament to replace D3 domain. The diameter of hFliC was computationally determined to be 35 nm. Negative stain TE micrographs confirmed resulting hFliC filaments had increased diameter compared to FliC filament. Referring to FIG. 2B, binding ELISA was performed using DENV2 specific monoclonal IgA antibody. hFliC showed binding to monoclonal IgA, compared to FliC alone, which showed no binding. Experiments were performed in triplicate (n=3), and error bars represent mean+/−SEM, Student's t-test was used to determine statistics, and a p<0.05 were considered statistically significant. Referring to FIG. 2C, immunogold particle labeling against DENV2 E specific IgG (primary antibody, isolated from 4G2 culture supernatant), was used to determine the placement of the DENV2 E portion relative to the rest of the filament. Negative stain transmission electron micrograph of immunogold labeling of WT FliC filaments (left panel), confirmed that no immunogold particles bound to WT filaments. Right panel is a negative stain transmission electron micrograph of immunogold labeling of hFliC filaments, which indicated that the immunogold particles primarily associate with the outside of the hFliC filament.

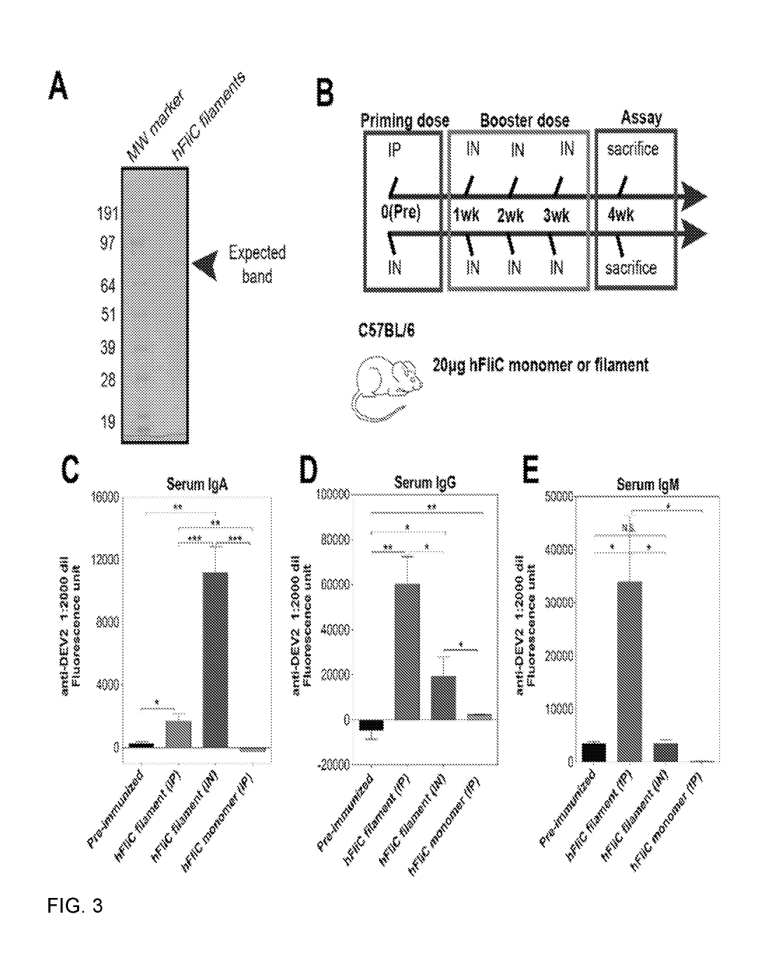

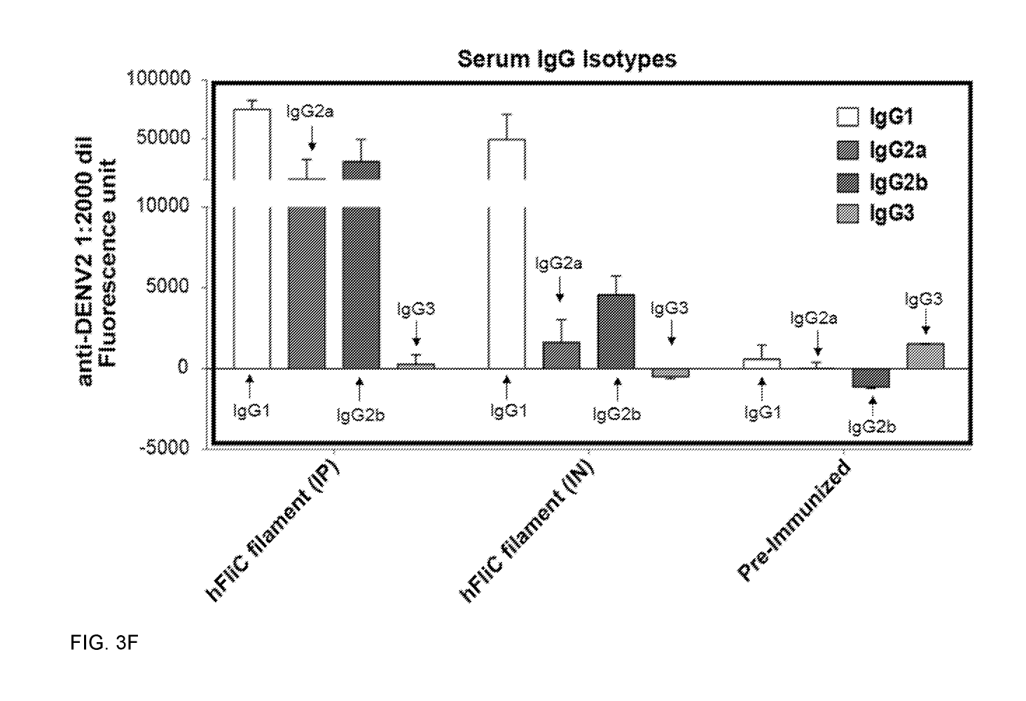

Referring to FIG. 3, humoral immune response in non-restricted mouse models is shown. Referring to FIG. 3A, purified hFliC protein was polymerized in high molar ammonium sulfate and resulting filaments were pelleted with high-speed centrifugation and resuspended in 150 mM PBS. Under denaturing conditions the major band was only that of the full-length hFliC. FIG. 3B is a schematic diagram of the in vivo immunization protocol used. The immunization period was five weeks, with four injections spaced a week apart. Priming injection at week zero was either administered IP or IN, and the three subsequent booster doses where all administered IN. Referring to FIG. 3C, ELISA scans show serum IgA response in WT C57BL/6J mice, assayed after the five-week immunization period. C57BL/6J mice given polymeric hFliC IN throughout the entire immunization exhibited a strong serum IgA response, while all other mice showed no IgA titer. Referring to FIG. 3D, ELISA scans for serum IgG response in WT C57BL/6J are shown, assayed after the five-week immunization period. C57BL/6J mice given polymeric hFliC IP showed a stronger IgG response compared to mice given monomeric hFliC IP. FIG. 3E shows ELISA scans for serum IgM response in WT C57BL/6J mice assayed after the five-week immunization period. Again, C57BL/6J mice receiving polymeric hFliC IP showed strongest IgM response. Referring to FIG. 3F, serum IgG isotypes were assayed in C57BL/6J mice, and mice given polymeric hFliC IP showed a robust IgG1 response. All groups in immunization protocols used an n=3-5 mice per group, and error bars represent the mean+/−SEM, and for statistical analyses, *-p<0.05; **-p<0.005; ***-p<0.0005. Values were reported as the fluorescence measured in 1:2000 serum dilutions, with background fluorescence values subtracted.

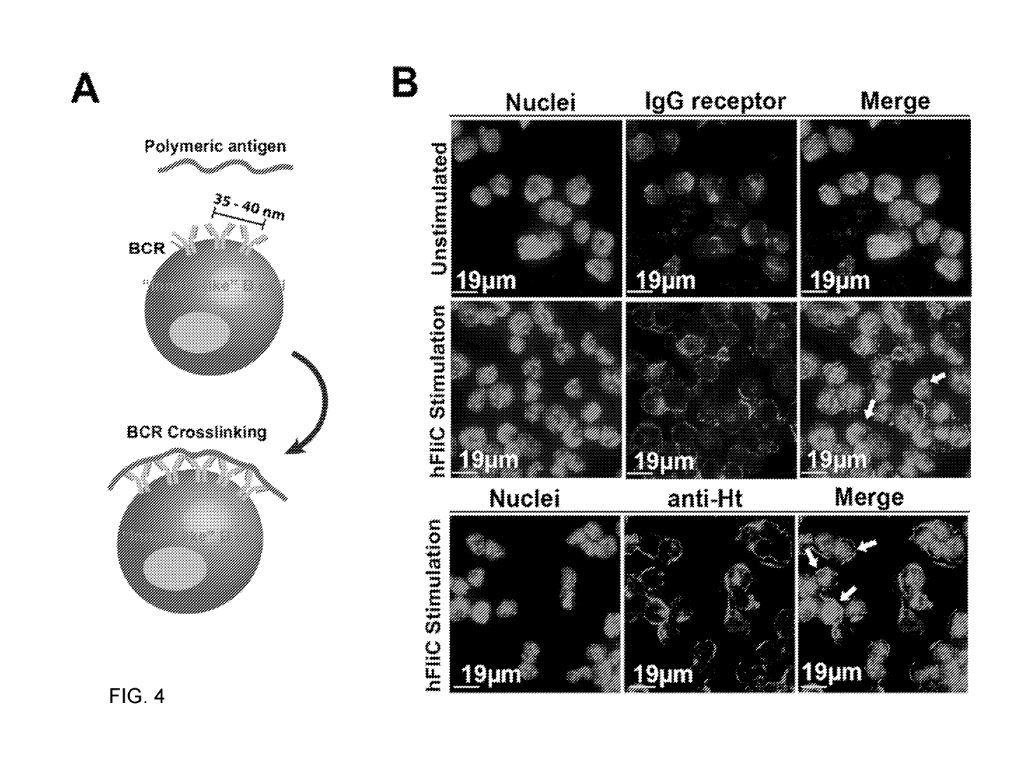

FIG. 4 shows that hybrid flagellin filaments are able to crosslink BCR in vitro. FIG. 4A is a schematic of how a TI immune response is generated. Polymeric antigens bind to B cell receptors and crosslink them. Once this crosslinking event occurs, the B cell will self-differentiate into plasma cells to produce antibodies. Referring to FIG. 4B, 4G2 B cell hybridoma (DENV2-specific IgG producing hybridoma) were stimulated with polymeric hFliC. Stimulated cells showed more organized expression of Ig receptors compared to unstimulated cells, indicating cells were crosslinked (top panel). This was further confirmed by staining with anti-Histag antibody.

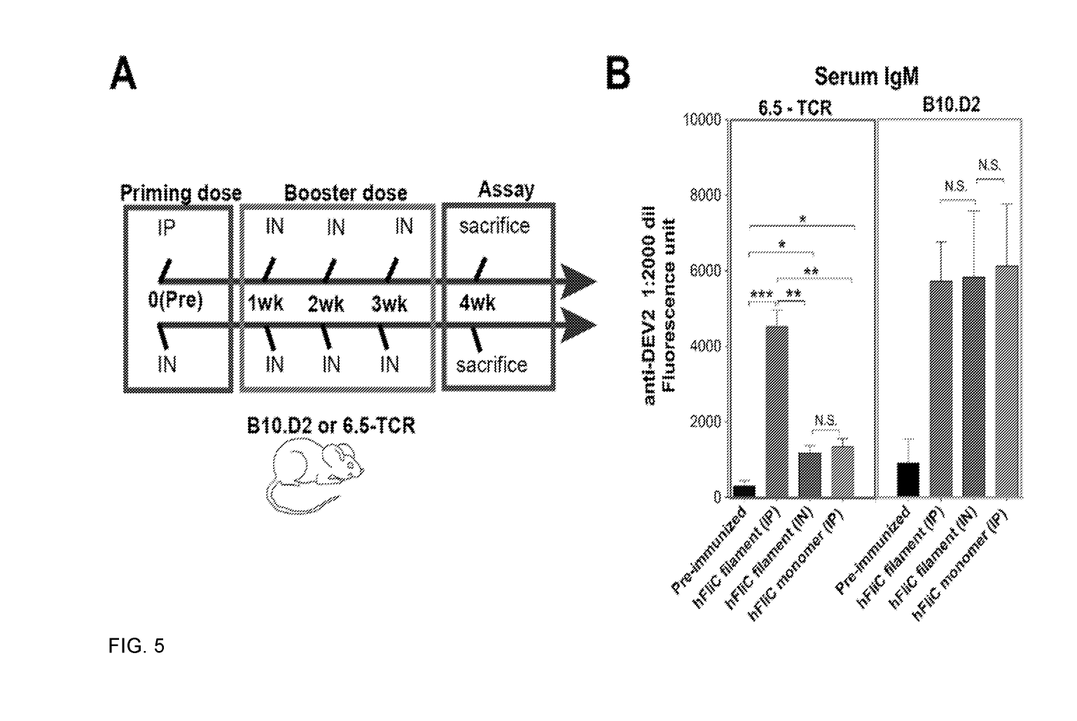

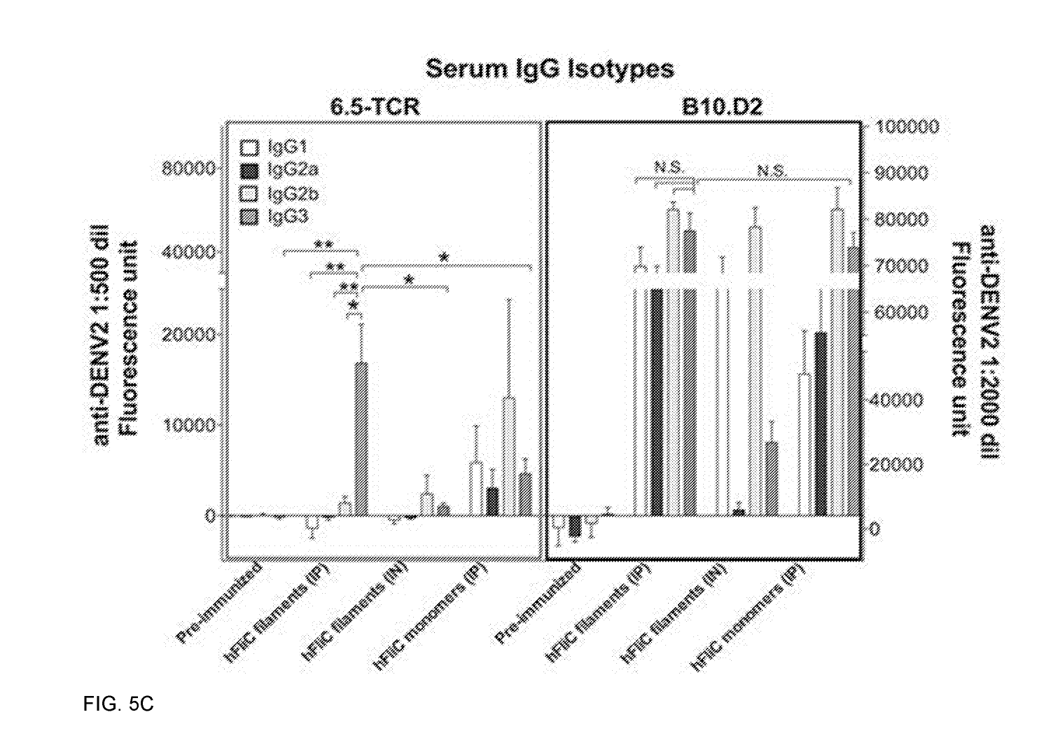

FIG. 5 shows humoral immune response in TI mouse model (6.5-TCR transgenic). FIG. 5A is a schematic diagram of in vivo immunization protocol used. The immunization period was five weeks, with four injections spaced a week apart. Priming injection at week zero was either administered IP or IN, and the three subsequent booster doses were administered (IN). FIG. 5B shows ELISA scans of serum IgM in 6.5-TCR and B10.D2 over five-week immunization period. Only mice given polymeric hFliC IP in the priming dose showed a serum IgM response. All groups B10.D2 mice immunized showed a strong IgM response. Statistics were determined by unpaired Student's t-test, p<0.05 were considered statistically significant, and error bars represent the mean+/−SEM. FIG. 5C shows ELISA scans of serum IgG isotypes in 6.5-TCR, and B10.D2 mice. 6.5-TCR mice given polymeric hFliC IP in the priming dose exhibited a specific IgG3 response, meanwhile B10.D2 mice showed broader subclass distribution (more balanced between Th1 and Th2). All groups in immunization protocols used an n=3-5 mice per group, and error bars represent the mean+/−SEM. Values were reported as the fluorescence measured in 1:2000 serum dilution in the case of IgM and IgG isotypes for B10.D2 immunized mice, and values were reported as the fluorescence measured in 1:500 serum dilution for IgG isotype for 6.5-TCR immunized mice, with background fluorescence values subtracted, and for statistical analyses, *-p<0.05; **-p<0.005; ***-p<0.0005. Arrows show that there was a specifically increased IgG3 response in 6.5-TCR mice to IP hFliC filament while IgG2b responses were higher to monomer, while by contrast, in B10.D2 mice IgG1 and IgG2a responses were strong to both IP hFliC filament and monomer.

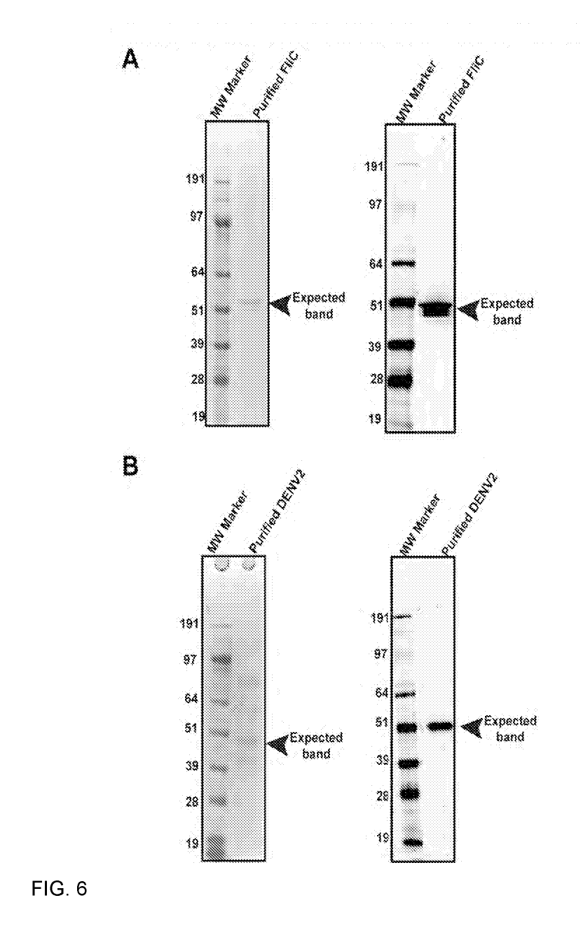

FIG. 6 shows the production of purification of recombinant FliC and DENV2. Referring to FIG. 6A, Coomassie (left panel) and Western blot against the C-terminal His tag were used to assess purity and size (56 kDa) of recombinantly produced FliC protein. Both Coomassie and Western blot confirmed FliC size as well as its relative purity, though some degradation was evident. FIG. 6B is an analysis to confirm DENV2 purity and size (50 kDa), which revealed that recombinantly produced DENV2 had appropriate size.

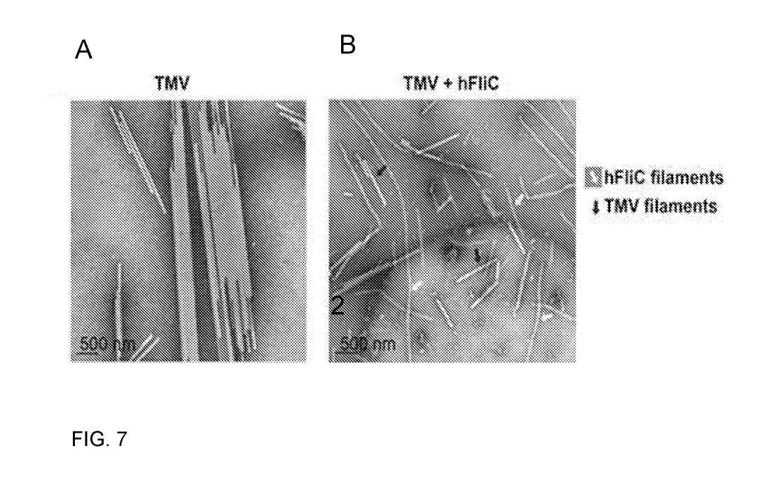

FIG. 7 shows a further characterization of hFliC filaments. FIG. 7A shows a negative stain transmission electron micrograph of TMV (tobacco mosaic virus), which was used as a positive control for contrast (high degree of contrast). FIG. 7B shows a negative stain transmission electron micrograph of hFliC filaments compared directly to TMV filaments. The micrograph indicates the inner core the hFliC filaments have a high degree of contrast, while the outer portion (i.e. DENV2) lacks contrast.

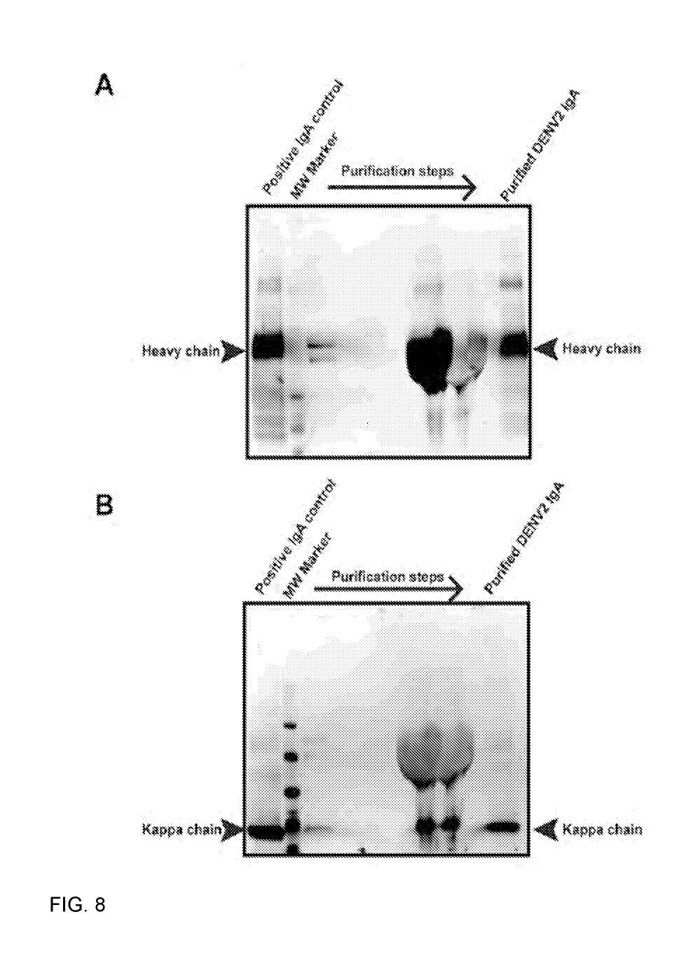

FIG. 8 shows recombinantly produced DENV2 specific monoclonal IgA. Positive IgA control used was purified mouse IgA, Kappa isotype control. Referring to FIG. 8A, Western blot probing for IgA heavy chain blot indicates that DENV2 monoclonal IgA has the anticipated size as compared to positive control. Referring to FIG. 8B, showing Western blot probing for IgA kappa (light) chain, again the blot indicates that DENV2 monoclonal IgA has the appropriate size as compared to positive control.

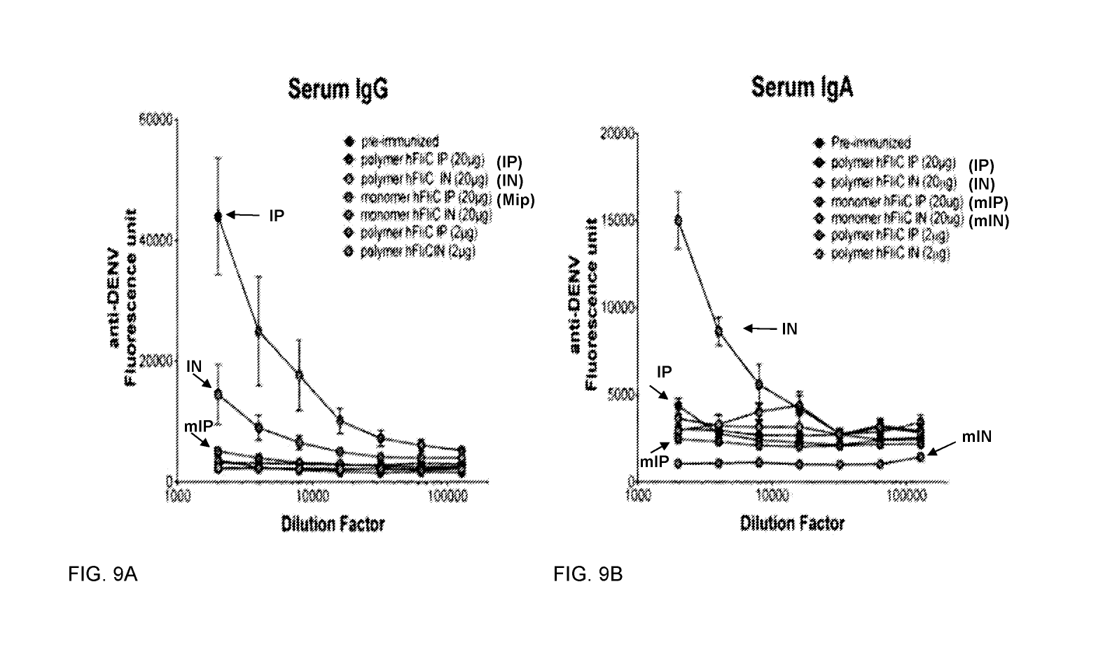

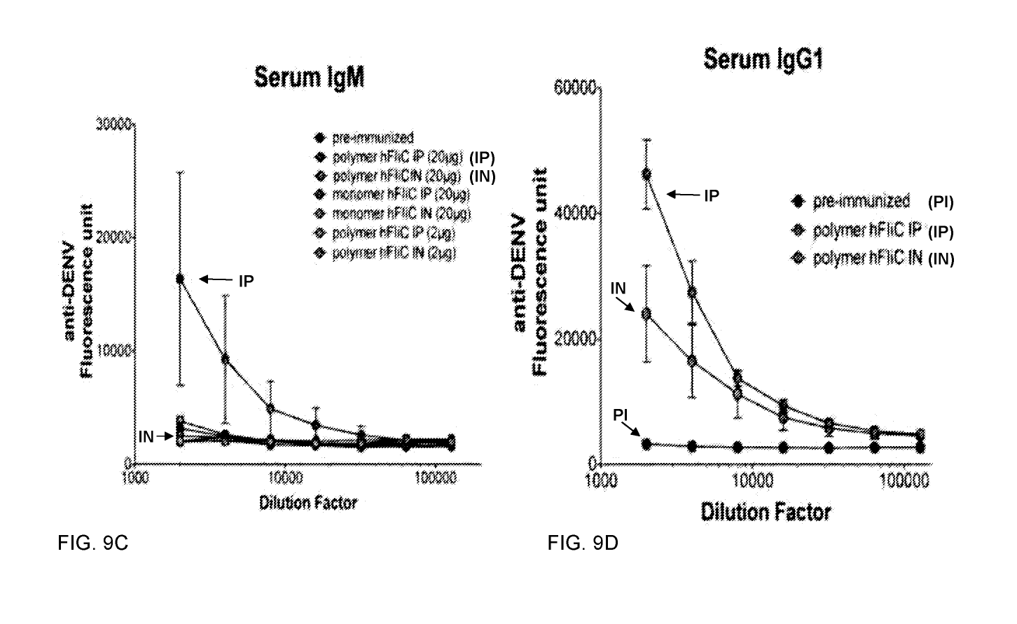

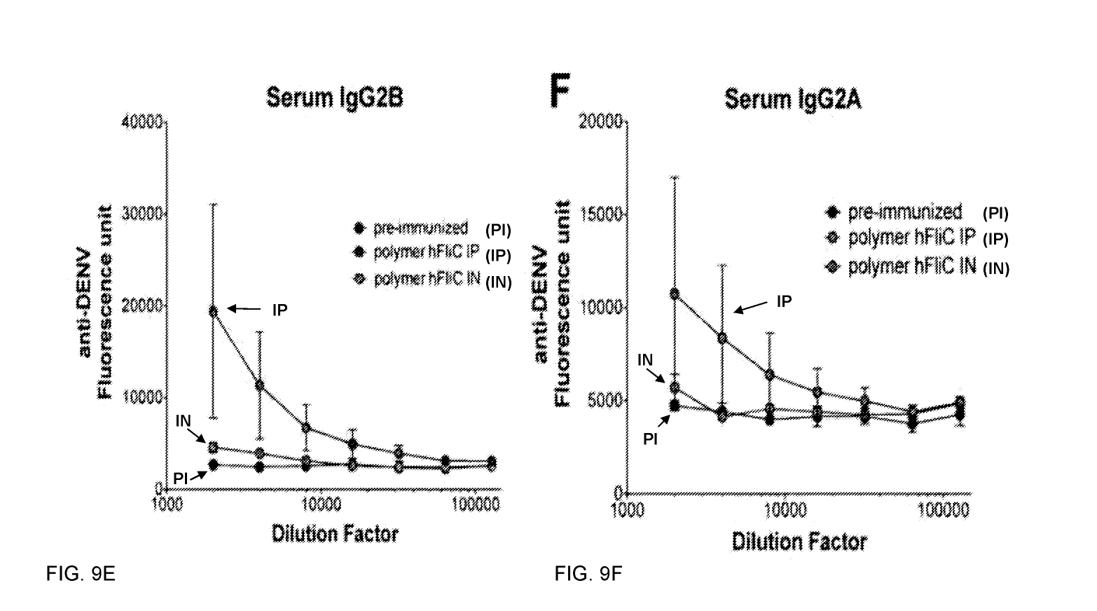

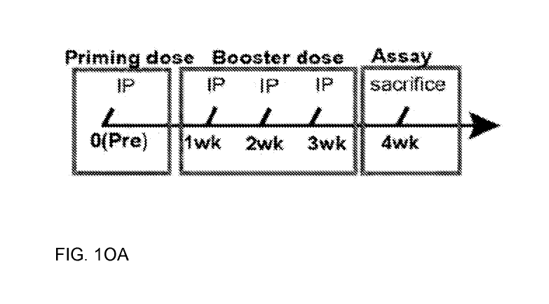

FIG. 9 provides ELISA curves from immunized C57BL/6J mice, with either 20 or 2 μg dose (which showed no response). All curves show the dilution series that was used. These values were subtracted from background fluorescence's and in most instances the 1:2000 dilution were used to make comparisons between groups. Same analysis was used for B10.D2 and 6.5-TCR immunized mice (but not shown). FIGS. 9A and 9B show results for serum IgG and IgA, respectively. FIGS. 9C and 9D show results for serum IgM and IgG1, respectively. FIGS. 9E and 9F show results for serum IgG2B and IgG2A, respectively.

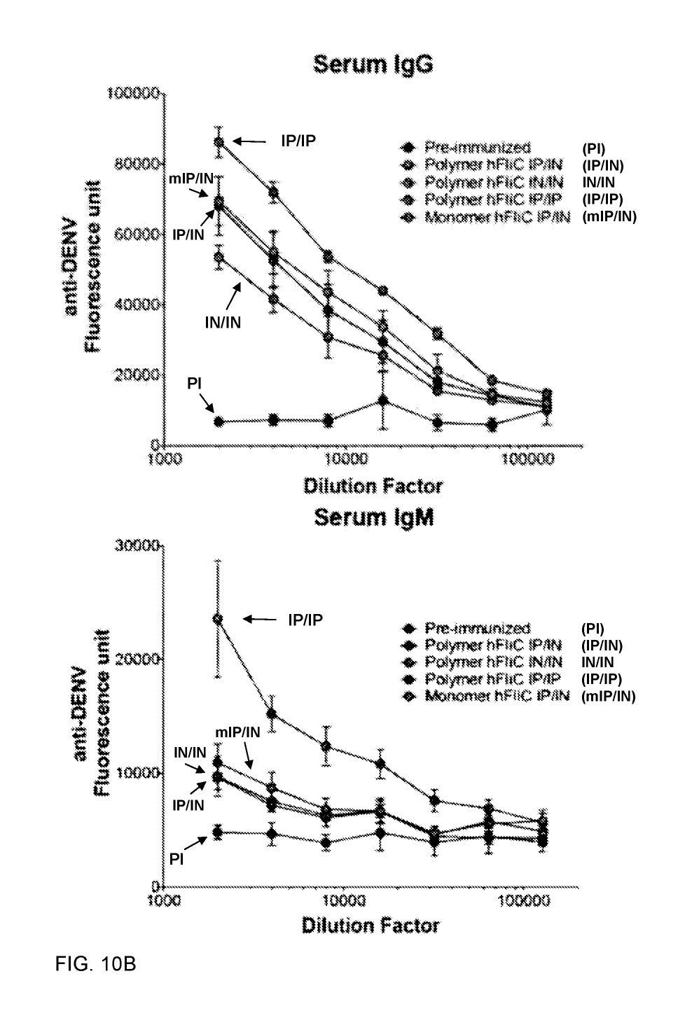

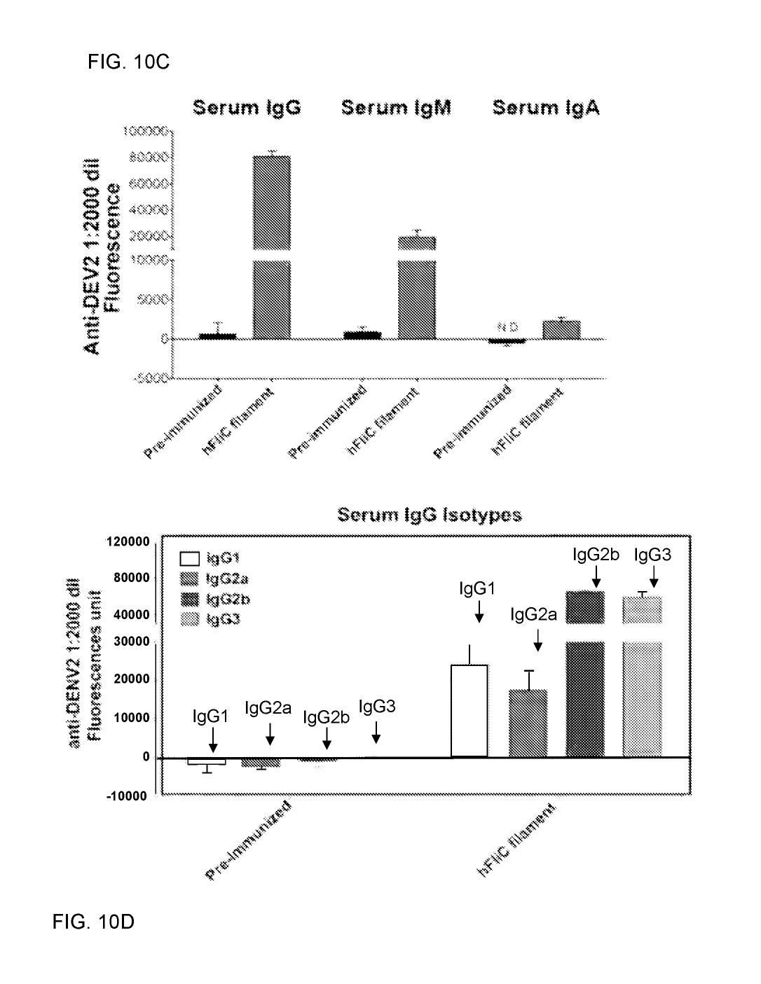

FIG. 10 provides results for B10.D2 mice immunized with hFliC administered via IP injections. FIG. 10A is a schematic of the immunization protocol that was used where IP injection was not only used in priming injection but also in all subsequent injections. B10.D2 mice given on hFliC filaments IP showed strongest IgG and IgM compared to all mice used in this study. FIG. 10B represents the raw dilution series, also comparing other immunization protocols, showing that the IP protocol induced the strongest IgM and IgG response. FIG. 10C represents the response after background had been subtracted. As shown in FIG. 10D, mice exhibited a broad subclass IgG isotype response.

### Results

Design and Characterization of hFliC Protein

To take advantage of the solvent exposed location of the D3 domain of FliC, this domain was replaced with a vaccine antigen domain, so that the surface of the polymerized hybrid protein filament would present polymeric vaccine antigen instead of flagellin epitopes. Thus, the flagellin D3 domain from residues 185 to 285 were removed and replaced with the extracellular domain of the DENV2 envelope protein (Dengue 2 E: FIG. 1A). DENV2 E is one of three structural proteins produced by the Dengue 2 virus (there are 4 identified Dengue serotypes, sharing ˜70% genome homology) and forms an icosahedral scaffold at the virion surface. DENV2 E mediates the fusion of virus to host cells and as such has been extensively studied for its antigenicity as a DENV2 vaccine [20]. In order to maintain the conformational independence of this domain, Gly-Ser flexible linkers were added to join FliC to the N- and C-terminus of DENV2 E. The resulting hybrid protein is therefore comprised of a central independent DENV 2 E protein domain, flanked by the two independent N- and C-terminal helical domains of FliC; folding of the E protein domain will help stabilize the helical domains of FliC, although further stabilization occurs in the polymerized filament when interactions between helical domains of the monomers interact. Finally, a C-terminal His₆-tag was added for production and purification purposes. The hybrid FliC protein (hFliC) was produced using a baculovirus expression system, purified (FIG. 1B), and subsequently assayed for appropriate structure and function. After His-tag enrichment, the hFliC band is clearly evident although comprising only about 14% of total protein; Western blot indicates a possible proteolytic cleavage near the N-terminus.

Circular dichroism (CD) was used to assess the secondary structure of purified hFliC protein (FIG. 1C), and compared to that of free FliC and DENV2. Quantification of percent secondary structure from the CD spectra revealed that FliC (protein size and purity confirmed: FIG. 6A) was largely α-helical, Meanwhile, DENV2 (protein size and purity confirmed: FIG. 6B) had a larger β-sheet region, consistent with previous published spectra, and was in good agreement with computationally determined secondary structure percentages based on crystallographic structures (Table 1: [21]). The hybrid hFliC exhibited a spectrum of mixed α-helical and β-sheet character (FIG. 1C). Taken together these data suggest that whole protein antigens can be inserted into FliC backbone without jeopardizing the native secondary structure of either protein.

TABLE 1
Quantification of secondary structure from CD spectra

| % of Secondary | | | |
|---|---|---|---|
| Structure | α-helix | β-Sheet | Random coil |
| FliC | | | |
| Computationally | 43.00 | 21.00 | 35.00 |
| determined¹ | | | |
| Experimentally | 71.10 | 7.20 | 21.70 |
| determined² | | | |
| DENV2 E protein | | | |
| Computationally | 7.00 | 48.00 | 55.00 |
| determined¹ | | | |
| Experimentally | 2.03 | 39.21 | 58.00 |
| determined² | | | |
| hFliC | | | |
| Experimentally | 56.00 | 18.40 | 25.60 |
| determined² | | | |

¹Calculated using Stride
²Calculated using k2d3

Characterization of hFliC Filaments

A hypothetical molecular model of the hFliC filament predicts an independent domain of DENV E at the surface of an intact 11-protofilament polymer (FIG. 2A). Accordingly, it was determined that the hFliC protein was able to form filaments in high molar ammonium sulfate or sodium citrate (FIG. 2A and data not shown), similar to the reported molar conditions for in vitro WT FliC polymerization [15]. To evaluate these filaments, negative stain transmission electron microscopy (TEM) was used. Morphometric analysis of the filaments revealed an increased diameter of 35 nm compared to WT FliC filaments (diameter of ˜20 nm [14]), consistent with the hypothetical molecular model of the hFliC filament (FIG. 2A). However, there is not sufficient resolution to confirm that the hFliC forms the prototypical 11-protofilament core structure as suggested by the hypothetical molecular model.

The inner core of the hFliC filaments exhibited a high degree of contrast, comparable to Tobacco Mosaic Virus (TMV) filaments (known to have high contrast) (FIG. 7A,B). However, the outer regions of the filaments displayed a low degree of contrast relative to the rest of the filament suggesting that individual DENV2 antigen domains were able to rotate freely at the surface of the filament (further confirmed via cryoelectron microscopy, data not shown). This flexibility might be beneficial to the avidity of the DENV2 antigenic domain of the polymeric hFliC vaccine.

To confirm that the DENV2 E domain retained its antigenicity, a DENV2 specific recombinant IgA monoclonal antibody (FIG. 8A,B) was used; this antibody was produced from sequences derived from the DENV2-specific 4G2 hybridoma because it was discovered that the original hybridoma produced mixed antibody complexes, not all being specific for DENV2 E protein. The recombinant IgA binding to control recombinant DENV2 E domain was tested, as well as in the hybrid hFliC protein and polymerized hFliC filament. In an ELISA assay, the DENV2 E hFliC was able to bind the 4G2 IgA (FIG. 2B, negative control FliC alone).

More importantly, it was confirmed that DENV2 E epitopes were available on the surface of the polymerized filament. This was confirmed by immunogold labeling of the hFliC filament using DENV2 specific IgG produced by the 4G2 hybridoma (FIG. 2C). The 4G2 antibody is known to bind/recognize an epitope region in the extremity of domain DII (important for membrane fusion) of the DENV2 E protein; based on the hFliC molecular model, this is the domain most solvent exposed in filament formation (FIG. 1A: [22,23]). Together, these data demonstrate 1) that the hFliC protein is able to form filaments in a similar manner to WT FliC, 2) the DENV2 E subunit inserted into the middle of FliC retains its conformational independence and 3) its antigenicity is retained and recognized by anti-DENV2 E-specific antibody.

In Vivo Activity of hFliC in TD Model

Since it was demonstrated that hFliC exhibited proper conformation and antigenicity in its polymerized form, testing of its ability to stimulate an immune response in vivo was performed. Using an immunization protocol similar to that used by Lo et al. [24] but in this case without the addition of any additional adjuvant (such as cholera toxin), the immunogenicity of monomeric and polymeric hFliC in wild type mice (C57BL/6J; FIG. 3B) was tested. A priming dose of hFliC filaments (FIG. 3A) or hFliC monomers was first administered, given either intraperitoneally (IP) or intranasally (IN), followed by three IN booster doses at one-week intervals for 3 additional weeks.

It was found that hFliC filaments induced a stronger antibody response than their monomeric counterparts in C57BL/6J WT mice (FIG. 3C-E and FIG. 9). IP administration of polymeric hFliC during the priming injection resulted in the highest serum IgG (total) and IgM responses (FIG. 3D,E and FIG. 9A,C). Mice given IN priming and booster injections of polymeric hFliC throughout the immunization protocol showed the strongest serum IgA response (FIG. 3C and FIG. 9B). Since different delivery routes gave different Ig responses in different tissues, we also tested whether these immunization protocols resulted in the production of different IgG subclasses (FIG. 3F). Thus, polymeric hFliC with either IP or IN priming induced strong serum IgG1 response, while the IP priming dose induced the best IgG2a and IgG2b response. In sum, the IN priming protocol induced both the best IgA and weakest IgG2a/b response, consistent with a Th2-type response. By contrast, the IP priming protocol induced the strongest IgG2 and IgM responses, though this would not be as easily categorized as a strict Th1-type response.

hFliC Filaments can Crosslink BCRs

TI responses rely on the ability of antigens to crosslink BCRs above a threshold number; this clustering promotes the T helper cell-independent differentiation of B cells into plasma cells (FIG. 4A). To determine whether the repetitive DENV2 E determinants displayed on hFliC filaments were capable of BCR crosslinking, they were tested on cells expressing receptors specific for the DENV2 E protein. The 4G2 B cell hybridoma expresses surface Ig receptors specific for DENV2 E protein, so these cells were incubated with hFliC filaments. Confocal micrographs of cytospin slides of unstimulated cells exhibited a randomized distribution of Ig receptors, while cells incubated with hFliC filaments exhibited a more organized distribution of the BCRs around the circumference of the cell (FIG. 4B, showing staining of surface Ig). This was further confirmed by staining with an anti-HisTag antibody (FIG. 4B), suggesting that the hFliC filament was in direct contact with the BCR receptors and these receptors had in fact been clustered by the hFliC filaments.

In Vivo Activity of hFliC Polymers in TCR Restricted Model

To test the potential for the polymeric vaccine to induce TI responses, in vivo immunization was performed in a T cell repertoire-restricted model (FIG. 5A). Here a T cell receptor transgenic mouse model was used (6.5-TCR, recognizing an influenza hemagglutinin peptide presented on MHCII I-E^{d}: [25,26]), in which nearly all CD4 T cells express the mono-specific hemagglutinin-specific antigen receptor. Allelic exclusion during thymic selection would prevent the development of T cells expressing any additional antigen receptors. This particular transgenic mouse model was chosen because the degree of allelic exclusion is more potent in this model compared to most other models, obviating the need for including additional targeted genes (e.g., Rag1) that would cripple the immune system. As a result, the monospecific CD4 T cell population only recognizes an antigen from influenza not seen under normal conditions, and so any CD4 T helper cells would be unable to provide cognate antigen-specific help to FliC- or DENV-specific B cells. Since a relatively normal number of T cells are still produced, all other components of the immune system, including organized lymphoid tissues, would still develop normally. Since the 6.5-TCR mice were on a B10.D2 background, B10.D2 mice were used as WT controls.

With an immunization protocol using hFliC filaments administered IP (priming dose), 6.5-TCR mice were still capable of producing a strong serum IgM response within the range of B10.D2 controls (within a two-fold dilution); no detectable levels of IgA were observed in any condition (FIG. 5B and FIGS. 10A-C). Interestingly, B10.D2 mice treated with polymeric hFliC in three IP doses exhibited the most robust IgM and IgG of any group of mice used in this study (FIG. 10D), raising the possibility that this protocol was particularly effective in targeting peritoneal B1 cells. Finally, we wanted to determine whether differences existed in the IgG isotypes produced in wild type B10.D2 or the TCR restricted (6.5-TCR) mice, as the IgG3 subclass has been previously identified as more prominent in TI antibody responses [9]. B10.D2 mice showed a robust Th1 serum IgG2b response, with a lower serum IgG3 response (FIG. 5C and FIG. 10D). Interestingly, although IgG titers were lower in the 6.5-TCR mice compared to B10.D2 mice due to the absence of cognate T cell help, 6.5-TCR mice given hFliC filament IP showed a significant IgG3 response (FIG. 5C).

### Discussion

Taken together, these data suggest that hFliC filaments are indeed capable of inducing a significant antibody response in a T cell restricted model. Development of vaccines with multivalent binding (clustering) has largely been accomplished by the linkage of two proteins in a linear fashion; however, this method has had limited success in certain diseases [27-29]. Thus, the reengineering of FliC by replacing its solvent exposed FliC D3 domain with a conformationally intact viral envelope protein domain may hold promise for an entirely new vaccine framework. This may be especially important in the development of vaccines for patients with less robust immune systems, including those in the early perinatal period, in people suffering from various T cell immunodeficiency disorders (HIV-AIDS, DiGeorge syndrome), patients undergoing chemotherapy, as well as elderly patients with sluggish adaptive immunity.

The increased potency of the hFliC relative to the monomeric form may relate to multiple factors. In the polymeric form the antigen may be more able to cross-link the antigen-specific B cell receptors. As a less soluble complex the filament may also be more easily taken up by antigen presenting cells for stimulation of T helper cells. In addition, the polymeric antigen may also have a greater potential to recruit the T helper-independent B-1 B cell population; this is evidenced by the strong IgM and IgG3 response in the 6.5-TCR mice when hFliC filaments were injected into the peritoneum, where B-1 B cells are dominant. More detailed studies on the IgM (and IgG3) response will be helpful in determining any benefit in recruiting B-1 B cells in protective immunity, as it may help recruit a distinctive antibody repertoire.

To date no licensed vaccine exists for DENV infection. This is complicated by the concern that sub-neutralizing levels of cross-reactive anti-DENV antibodies might result in more severe disease (i.e., Dengue Hemorrhagic Fever; [30-34]. Thus, it is crucial to develop a vaccine framework that is equally effective against all DENV serotypes. Live attenuated tetravalent viruses have shown only limited success in both non-human primates and humans, but it has been difficult to establish a proper balance between immunogenicity and adverse reactions with these formulations [35,36]. In addition, while recombinant DENV subunit vaccines can induce neutralizing titers in mice when co-administered with adjuvant [37], these were found to be less efficacious. The approach described here of reengineering FliC to replace its solvent exposed FliC D3 domain with antigenic determinants allows for combining the benefits of the immunological adjuvant effects of flagellin with the added immunogenicity of polymeric vaccine antigens. Moreover, independent replacement of the FliC D3 domain with envelope proteins from each of the four DENV serotypes, and then producing mixed filaments with stoichiometric amounts of each hFliC protein could produce a universal DENV vaccine with balanced broadly neutralizing capabilities (i.e. against all serotypes). This strategy may overcome the problem noted in a recent trial with a tetravalent recombinant vaccine, in which the response to one serotype was much lower than the response to the other serotypes [38].

### CONCLUSIONS

The native FliC structure was reengineered by replacing the FliC D3 domain to contain a conformationally intact DENV2 E protein domain, without compromising either protein's structure. Moreover, this hybrid protein could be polymerized to produce filaments capable of stimulating a humoral antibody response; filaments were found to be more potent induces of antibody responses than monomers. More importantly, these hybrid protein filaments were capable of crosslinking B cell antigen receptors in vitro and accordingly were also shown to induce significant antibody responses even in a T cell repertoire-restricted transgenic mouse model. In sum, this study lays the groundwork for the use of FliC as a vaccine scaffold for development of novel vaccines for use even in immunodeficiency settings.

### REFERENCES

The following publications are incorporated by reference herein in their entirety:
1. Medzhitov R, Janeway C A, Jr. Innate immunity: impact on the adaptive immune response. Current opinion in immunology. 1997; 9:4-9.
2. Parker D C. T cell-dependent B cell activation. Annual review of immunology. 1993; 11:331-60.
3. Mosmann T R, Cherwinski H, Bond M W, Giedlin M A, Coffman R L. Two types of murine helper T cell clone. I. Definition according to profiles of lymphokine activities and secreted proteins. Journal of immunology. 1986; 136:2348-57.
4. Baumgarth N. The double life of a B-1 cell: self-reactivity selects for protective effector functions. Nature reviews Immunology. 2011; 11:34-46.
5. Balazs M, Martin F, Zhou T, Kearney J. Blood dendritic cells interact with splenic marginal zone B cells to initiate T-independent immune responses. Immunity. 2002; 17:341-52.
6. Dintzis H M, Dintzis R Z, Vogelstein B. Molecular determinants of immunogenicity: the immunon model of immune response. Proc Natl Acad Sci USA. 1976; 73:3671-5.
7. Dintzis R Z, Okajima M, Middleton M H, Greene G, Dintzis H M. The immunogenicity of soluble haptenated polymers is determined by molecular mass and hapten valence. Journal of immunology. 1989; 143:1239-44.
8. Bachmann M F, Zinkernagel R M. Neutralizing antiviral B cell responses. Annual review of immunology. 1997; 15:235-70.
9. Mongini P K A, Stein K E, Paul W E. T-Cell Regulation of IgG Subclass Antibody-Production in Response to T-Independent Antigens. Journal of Experimental Medicine. 1981; 153:1-12.
10. Macpherson A J, Gatto D, Sainsbury E, Harriman G R, Hengartner H, Zinkernagel R M. A primitive T cell-independent mechanism of intestinal mucosal IgA responses to commensal bacteria. Science. 2000; 288:2222-6.
11. Vos Q, Lees A, Wu Z Q, Snapper C M, Mond J J. B-cell activation by T-cell-independent type 2 antigens as an integral part of the humoral immune response to pathogenic microorganisms. Immunological reviews. 2000; 176:154-70.
12. Stein K E. Thymus-independent and thymus-dependent responses to polysaccharide antigens. Journal of infectious diseases. 1992; 165 Suppl 1:S49-52.
13. Sanders C J, Yu Y, Moore D A, 3rd, Williams I R, Gewirtz A T. Humoral immune response to flagellin requires T cells and activation of innate immunity. Journal of immunology. 2006; 177:2810-8.
14. Yonekura K, Maki-Yonekura S, Namba K. Complete atomic model of the bacterial flagellar filament by electron cryomicroscopy. Nature. 2003; 424:643-50.
15. Wakabayashi K, Hotani H, Asakura S. Polymerization of Salmonella flagellin in the presence of high concentrations of salts. Biochimica et biophysica acta. 1969; 175:195-203.
16. Hayashi F, Smith K D, Ozinsky A, Hawn T R, Yi E C, Goodlett D R, et al. The innate immune response to bacterial flagellin is mediated by Toll-like receptor 5. Nature. 2001; 410:1099-103.
17. Smith K D, Andersen-Nissen E, Hayashi F, Strobe K, Bergman M A, Barrett S L, et al. Toll-like receptor 5 recognizes a conserved site on flagellin required for protofilament formation and bacterial motility. Nature immunology. 2003; 4:1247-53.
18. Zhao Y, Yang J, Shi J, Gong Y N, Lu Q, Xu H, et al. The NLRC4 inflammasome receptors for bacterial flagellin and type III secretion apparatus. Nature. 2011; 477:596-600.
19. Muskotal A, Seregelyes C, Sebestyen A, Vonderviszt F. Structural basis for stabilization of the hypervariable D3 domain of Salmonella flagellin upon filament formation. Journal of molecular biology. 2010; 403:607-15.
20. Modis Y, Ogata S, Clements D, Harrison S C. Structure of the dengue virus envelope protein after membrane fusion. Nature. 2004; 427:313-9.
21. Vonderviszt F, Kanto S, Aizawa S, Namba K. Terminal regions of flagellin are disordered in solution. Journal of molecular biology. 1989; 209:127-33.
22. Henchal E A, Gentry M K, McCown J M, Brandt W E. Dengue virus-specific and flavivirus group determinants identified with monoclonal antibodies by indirect immunofluorescence. The American journal of tropical medicine and hygiene. 1982; 31:830-6.
23. Rajamanonmani R, Nkenfou C, Clancy P, Yau Y H, Shochat S G, Sukupolvi-Petty S, et al. On a mouse monoclonal antibody that neutralizes all four dengue virus serotypes. The Journal of general virology. 2009; 90:799-809.
24. Lo D D, Ling J, Eckelhoefer A H. M cell targeting by a Claudin 4 targeting peptide can enhance mucosal IgA responses. BMC biotechnology. 2012; 12:7.
25. Kirberg J, Baron A, Jakob S, Rolink A, Karjalainen K, von Boehmer H. Thymic selection of CD8+ single positive cells with a class II major histocompatibility complex-restricted receptor. The Journal of experimental medicine. 1994; 180:25-34.
26. Degermann S, Reilly C, Scott B, Ogata L, von Boehmer H, Lo D. On the various manifestations of spontaneous autoimmune diabetes in rodent models. European journal of immunology. 1994; 24:3155-60.
27. Dormitzer P R, Ulmer J B, Rappuoli R. Structure-based antigen design: a strategy for next generation vaccines. Trends in biotechnology. 2008; 26:659-67.
28. Czerkinsky C, Russell M W, Lycke N, Lindblad M, Holmgren J. Oral administration of a streptococcal antigen coupled to cholera toxin B subunit evokes strong antibody responses in salivary glands and extramucosal tissues. Infection and immunity. 1989; 57:1072-7.
29. Fox C B, Kramer R M, Barnes V L, Dowling Q M, Vedvick T S. Working together: interactions between vaccine antigens and adjuvants. Therapeutic advances in vaccines. 2013; 1:7-20.
30. Brandt W E, McCown J M, Gentry M K, Russell P K. Infection enhancement of dengue type 2 virus in the U-937 human monocyte cell line by antibodies to flavivirus cross-reactive determinants. Infection and immunity. 1982; 36:1036-41.
31. Halstead S B, O'Rourke E J. Antibody-enhanced dengue virus infection in primate leukocytes. Nature. 1977; 265:739-41.
32. Dejnirattisai W, Jumnainsong A, Onsirisakul N, Fitton P, Vasanawathana S, Limpitikul W, et al. Cross-reacting antibodies enhance dengue virus infection in humans. Science. 2010 May 7; 328(5979):745-8.
33. Zellweger R M, Prestwood T R, Shresta S. Enhanced infection of liver sinusoidal endothelial cells in a mouse model of antibody-induced severe dengue disease. Cell Host Microbe. 2010 Feb. 18; 7(2):128-39.
34. Balsitis S J, Williams K L, Lachica R, Flores D, Kyle J L, Mehlhop E, et al. Lethal antibody enhancement of dengue disease in mice is prevented by Fc modification. PLoS Pathog. 2010; 6(2): e1000790.
35. Guirakhoo F, Pugachev K, Zhang Z, Myers G, Levenbook I, Draper K, et al. Safety and efficacy of chimeric yellow Fever-dengue virus tetravalent vaccine formulations in nonhuman primates. Journal of virology. 2004; 78:4761-75.
36. Simasathien S, Thomas S J, Watanaveeradej V, Nisalak A, Barberousse C, Innis B L, et al. Safety and immunogenicity of a tetravalent live-attenuated dengue vaccine in flavivirus naive children. The American journal of tropical medicine and hygiene. 2008; 78:426-33.
37. Sun W, Nisalak A, Gettayacamin M, Eckels K H, Putnak J R, Vaughn D W, et al. Protection of Rhesus monkeys against dengue virus challenge after tetravalent live attenuated dengue virus vaccination. The Journal of infectious diseases. 2006; 193:1658-65.
38. Sabchareon A, Wallace D, Sirivichayakul C, Limkittikul K, Chanthavanich P, Suvannadabba S, et al. Protective efficacy of the recombinant, live-attenuated, CYD tetravalent dengue vaccine in Thai schoolchildren: a randomised, controlled phase 2b trial. Lancet. 2012; 380:1559-67.
39. Shresta S, Sharar K L, Prigozhin D M, Beatty P R, Harris E. Murine model for dengue virus-induced lethal disease with increased vascular permeability. J Virol. 2006 October; 80(20):10208-17.
40. Marti-Renom M A, Stuart A C, Fiser A, Sanchez R, Melo F, Sali A. Comparative protein structure modeling of genes and genomes. Annual review of biophysics and biomolecular structure. 2000; 29:291-325.
41. Kuroda H. Polymerization of Salmonella, Proteus and Bacillus flagellins in vitro. Biochimica et biophysica acta. 1972; 285:253-67.
42. Liew C G, Draper J S, Walsh J, Moore H, Andrews P W. Transient and stable transgene expression in human embryonic stem cells. Stem cells. 2007; 25:1521-8.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of inducing a T cell-independent (TI) immune response in a subject, comprising
administering to the subject an immunogenic amount of a filament comprising one or more hybrid flagellin proteins, each hybrid protein comprising a heterologous polypeptide antigen and a portion of a FliC flagellin protein, whereby an TI immune response is generated against the antigen.

2. The method of claim 1, wherein the antigen replaces a whole or a part of FliC flagellin protein variable domain D3.

3. The method of claim 1, wherein the antigen comprises an amino acid sequence from a pathogenic virus, bacteria, fungus, protozoan parasite, or helminth parasite, or a combination thereof.

4. The method of claim 1, wherein the antigen comprises a protective epitope.

5. The method of claim 1, wherein the filament comprises two or more hybrid flagellin proteins, each hybrid protein comprising a different heterologous polypeptide antigen.

6. The method of claim 5, wherein the different heterologous polypeptide antigen is a polypeptide antigen from a different serotype of a pathogenic virus.

7. The method of claim 1, wherein the antigen is a viral envelope protein.

8. The method of claim 1, wherein the FliC flagellin protein is from S. Typhimurium.

9. The method of claim 1, wherein the TI immune response comprises production of IgM and/or IgG3.

10. The method of claim 1, wherein a T cell-dependent (TD) response is also generated against the antigen.

11. The method of claim 1, wherein the subject is a human or other mammal.

12. The method of claim 11, wherein the subject is immunocompromised.

13. The method of claim 11, wherein the subject is elderly or a neonate.

14. The method of claim 1, wherein the one or more hybrid flagellin proteins is administered with an adjuvant.

15. A vaccine composition, comprising an immunogenic amount of a filament comprising one or more hybrid flagellin proteins, each hybrid protein comprising a heterologous polypeptide antigen and a portion of a FliC flagellin protein.

16. The vaccine composition of claim 15, further comprising an adjuvant.
